(19) United States
(12) Patent Application Publication
(10) Pub. No.: US 2012/0095595 A1
(43) Pub. Date: Apr. 19, 2012

(54) PROCESS OF MAKING CEREAL, PULSE AND LENTIL ADMIXTURE

(76) Inventor: SUBRAMANIAN KRISHNAN, SANJOSE, CA (US)

(21) Appl. No.: 12/905,055

(22) Filed: Oct. 14, 2010

(51) Int. Cl. G05B 19/418; B67D 7/08; G01N 33/02; B02C 23/00; A21D 10/04; A21D 8/04

(52) U.S. Cl. 700/248; 426/496; 426/18; 426/231; 241/33; 222/52; 901/7

(57) ABSTRACT

A process and a method for automatically and/or semi automatically making a cereal and a pulse batter having an optimal consistency is described. The method and process also includes the steps for dispensing and storage for distribution. The cereal and pulse are soaked, ground and mixed together, spices added and allowed to ferment at an ambient temperature for an adequate time. The batter is packaged and stored in such a way that the batter's shelf life is increased. Expiration date is labeled on the container so that it can be monitored by the user. The process also provides a sterile way of preparing the various combinations of cereal and pulse as a batter. The optimal fermentation provides consistent delivery of taste. Packaging techniques provide appropriate shipping conditions.

### FIELD OF TECHNOLOGY

This disclosure relates generally to a method and a process to make an admixture, using a cereal batter, a pulse and a lentil batter in a specific proportion, having an optimal consistency for efficient processing to a product.

### BACKGROUND

Cereal and lentil batter is used in producing pancake like edibles and steamed cake edibles in Southern India. Custom production of combination of different cereal and lentil are done to produce a batter that can be used for at least three or four days. The vagaries of temperature fluctuation, grinding duration, differences in types of cereal and lentil variety create a challenge to produce uniform batter. In the summer heat the fermentation process is very quick and it leads to sourness of the batter and pungent tasting batter. In colder months there is insufficient fermentation and leads to flat batter with no taste.

The consistency of the batter also varies from batch to batch. There are no means to maintain uniform consistency of the batter. Uniform consistency of the batter would enable the supplier to produce predictable batter quality batch after batch and allow the end user to use the batter without modification. The nutritional values may be stabilized and optimal per unit consumption may be predicted for the user.

Furthermore, the apparatus is currently available in the market to make cereal and lentil batter. They are bulky and rotate at a constant speed. The users have to monitor the consistency and then remove the batter to mix them together. In addition, some batter making apparatus require manual tilting during its operation, adding to the complexity for bulk production. The method is laborious and time consuming. Furthermore, the apparatus size used for higher quantity renders the apparatus to be disparate, bulky for use and difficult to clean and transfer one batter and mix it with another batter to make an admixture.

### SUMMARY

The invention discloses a method, a process and an apparatus for making admixture using at least two ingredients in combination of batters such as a cereal and a lentil, a cereal and a cereal, and a lentil and a lentil.

In one embodiment, a semi-automated method of making the admixture is disclosed. Network controlled raw material processing, prepared material processing and packaging and shipping are performed.

In one embodiment, optimal proportion of raw materials such as a cereal and a lentil to be used are shown. In another embodiment, automatic optimal grinding speed, time and batter consistency measurement is described.

In another embodiment, optimal soaking conditions for cereal and lentil are shown. Once the soaking period is over it may be transferred to grinding station for individually grinding till optimal consistency is achieved. In another embodiment, once the optimal consistency is reached for cereal batter and lentil batter are combined and appropriate spices are added.

In another embodiment, the consistency measurement is exceeding the given time one may monitor the raise in temperature for the batter and cooling procedure may be adopted to prevent premature fermentation of the cereal and lentil admixture.

In another embodiment, the spiced mixed batter is fermented at a constant temperature and the end point is measured by ascertaining the batter level raise. Once the fermentation is completed, the batter is stirred to remove the excess air, stored in the cold chamber till they are ready to be packed. In another embodiment, packing is done using labeled containers and a little hole is made on the sealing material to let any further gas build up during transportation.

In one embodiment, the apparatus may be configured to dispense ingredients, soak the ingredients, mix the ingredients, ferment the mixture based on the input obtained from internal monitoring sensors and/or inputs given by the user. The apparatus may be capable of processing either a single component at a time or simultaneously processing one component while other is being processed. The apparatus may not require continuous monitoring by the user as the apparatus is configured to generate alert for the user if intervention is required.

The apparatus in one embodiment may be controlled by a computer. In another embodiment, sensors may be used to measure several parameters such as temperature, consistency of the admixture, pressure and weight. In another embodiment, several modules may be used using the computer to control the entire assembly process including but not limited to dispensing, weighing, movement of the container from one location to another and consistency control module.

The methods, systems, and apparatuses disclosed herein may be implemented in any means for achieving various aspects, and may be executed in a form of a machine-readable medium embodying a set of instructions. Other features will be apparent from the accompanying drawings and from the detailed description that follows.

### BRIEF DESCRIPTION OF THE DRAWINGS

Example embodiments are illustrated by way of example and not limitation in the figures of the accompanying drawings, in which like references indicate similar elements and in which:

FIG. 1 is a system view illustrating generation of an automated admixture process according to one embodiment.

FIG. 2 is a flow chart illustrating an edible material processing according to one embodiment.

FIG. 3 is a flow chart illustrating a prepped material processing, according to one embodiment.

FIG. 4 is a flow chart illustrating a packaging and shipping processes, according to one embodiment.

FIG. 5 illustrates an automation module 500 implemented the manufacturing unit as illustrated in FIG. 1, according to one embodiment.

FIG. 6 is a flow diagram illustrating a generation of admixture, according to one embodiment.

FIG. 7 is a perspective view illustrating a grinding process 112 according to one embodiment.

FIG. 8 illustrates various phases of preparing the admixture, according to one embodiment.

FIG. 9 is a diagrammatic system view 900 of a data processing system in which any of the embodiments disclosed herein may be performed, according to one embodiment.

FIG. 10 is a process 101 illustrating soaking and grinding operation 112 according to one embodiment.

FIG. 11 is a continuation of the process 101 illustrating additional operations according to one embodiment.

FIG. 12 illustrates a distribution management 1250, according to one embodiment.

Other features of the present embodiments will be apparent from the accompanying drawings and from the detailed description that follows.

### DETAILED DESCRIPTION

Example embodiments, as described below, may be used to provide a method and a process to make an admixture, using a cereal batter and a lentil batter in a specific proportion, having an optimal consistency for efficient processing to a product. Although the present embodiments have been described with reference to specific example embodiments, it will be evident that various modifications and changes may be made to these embodiments without departing from the broader spirit and scope of the various embodiments.

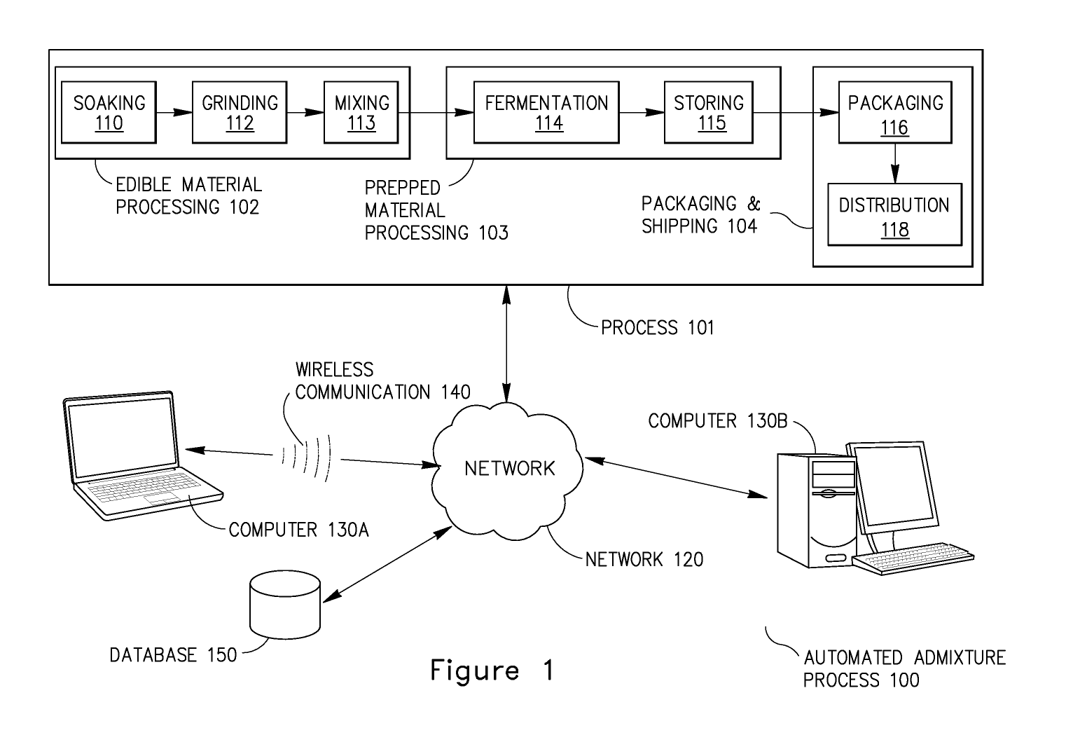

FIG. 1 is a system view illustrating generation of an automated admixture process 100 according to one embodiment. Particularly, the automated admixture process 100 includes process 101, computer 130A-B, network 120 and wireless communication 140. Process 101 in turn comprises of edible material processing 102, prepped material processing 103, packaging and shipping 104 according to one embodiment. The automated admixture process 100 is completely controlled through a computing device such as a computer or a programmable logic controller device. The automated admixture process may be controlled through automation software designed thereof for generating an admixture. The automated admixture process may be semi automated and a few steps may be performed manually. The computing devices that controls the automated admixture process 100 may be located in proximity with the manufacturing unit or in at a remote location.

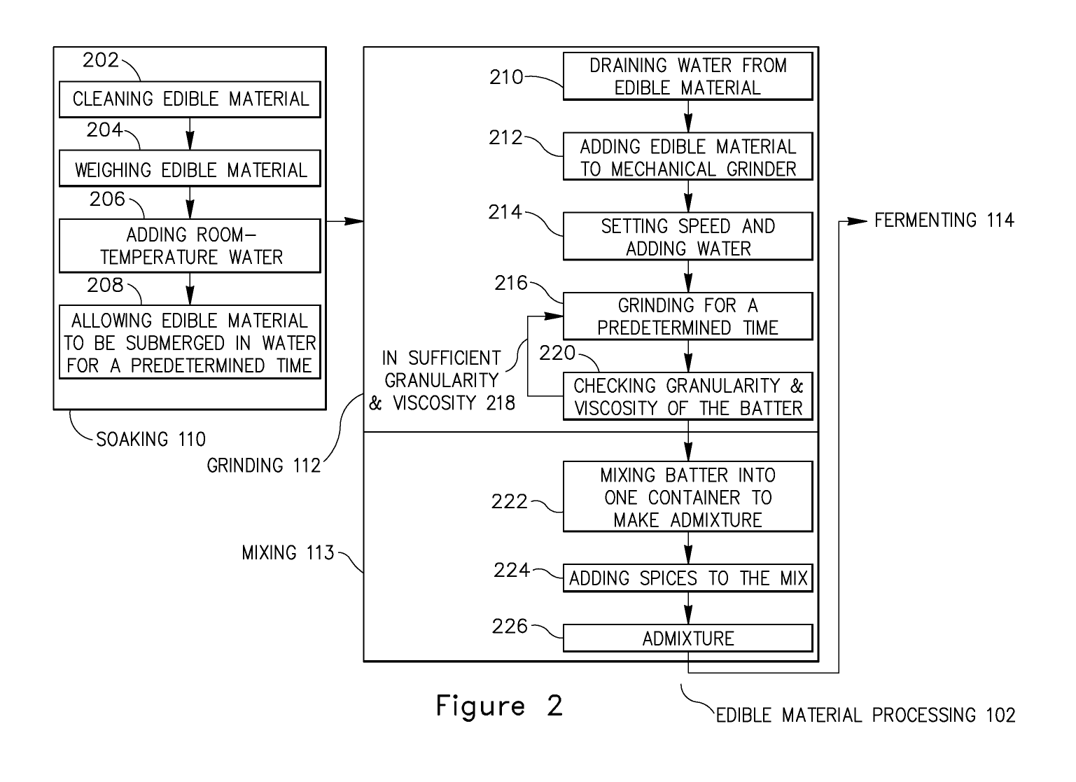

The computing devices such as a computer 130A and/or the computer 130B may be the computing devices for implementing an automated admixture process 100 according to one example embodiment. The manufacturing unit may be coupled to the computing device (e.g., the computer 130A and the computer 130B) through a network 120. The network 120 may be a wired network or a wireless network. The computing devices may be coupled to the network through the wired communication or through wireless communication 140. The manufacturing unit includes edible material processing unit, the prepped material processing unit and packaging and shipping unit. The edible material processing unit may include equipments required for processing the edible material. The edible material processing 102 may include a soaking process 110, grinding process 112 and the mixing process 113 according to one embodiment. A database 150 may be used for storing all the information such as weights, names, quantities and inventory. The edible material processing 102 is further explained in FIG. 2.

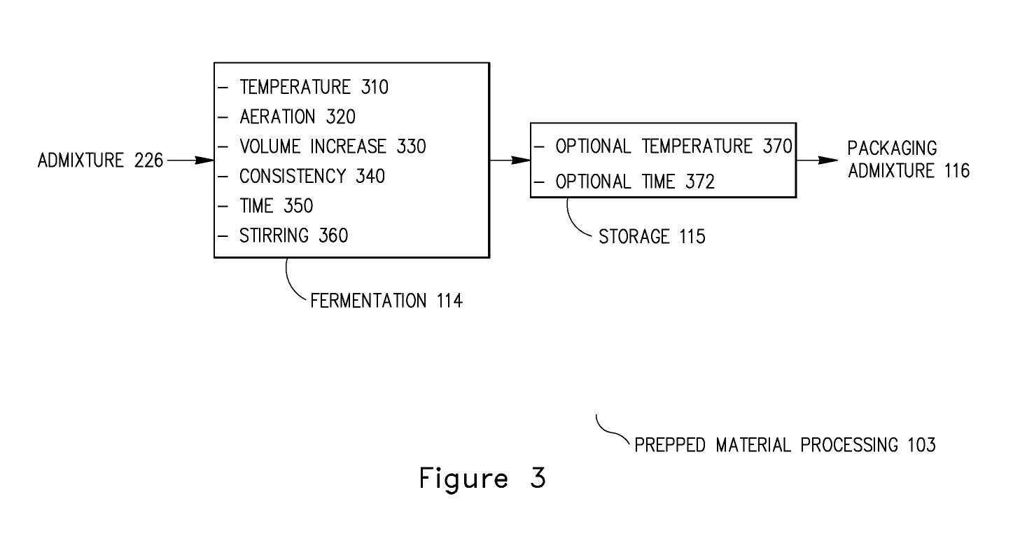

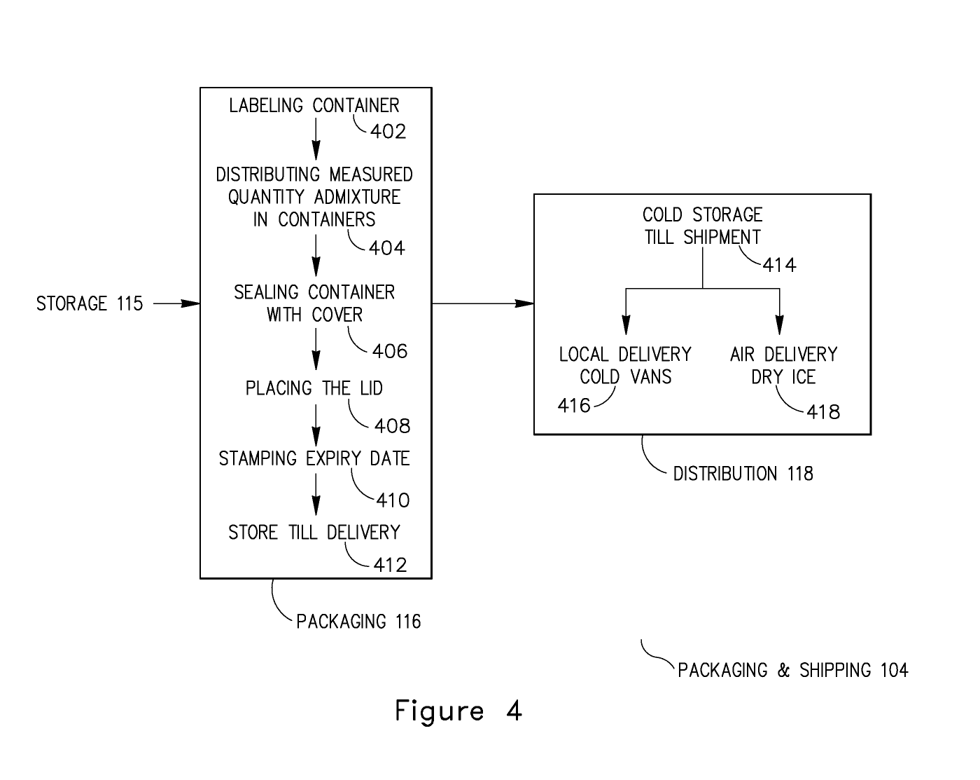

The prepped material processing unit includes specific equipments for preparing a final product. The prepped material processing 103 may include fermentation process 114, storing process 115 according to one embodiment. The prepped material processing 103 is explained in FIG. 3. The packaging and shipping unit may include packaging unit and distribution unit performing a packaging process 116 and a distribution process 118. The packaging and shipping 104 processes in explained in FIG. 4.

FIG. 2 is a flow chart illustrating an edible material processing 102 according to one embodiment. The soaking process 110 includes steps as described below. In step 202, edible materials for preparing a batter may be cleaned. For example, the edible materials may be cleaned by washing the edible materials in water repeatedly. In addition, the standard procedures including removal of minute earthly materials or contaminants such as small stones may be performed to render the edible materials ready for preparation of batter. In step 204, each of the edible materials may be weighed separately. Each of the weighed edible materials may be places in separate containers. In one embodiment, the edible material may include, but not limited to cereal, lentil, pulses, beans and the like. Cereal may be rice, wheat, semolina, barley, maize, oats, rye, sorghum, quinoa, amaranth, varieties of rice such as parboiled rice, wild rice, brown rice etc. Pulses may be black gram, red gram, mung bean, chickpea, bengal gram etc. In step 206, an appropriate amount of room temperature water based on weight and type of the edible material may be added. For example, the water requirement for soaking rice may be more than the volume of water required for soaking lentil. In step 208, each of the edible materials may be allowed to be submerged in water for a predetermined duration of time (30 minutes to 3 hours) in separate containers. After soaking the edible material for a predetermined duration of time the grinding process 112 may be initiated. Prior to the grinding process the soaked edible material may be cleaned using a contaminant removal machine to remove the unclean water and other solid contaminants present in the water layer.

In one embodiment, the grinding 112 process is as described below. In step 210, the liquid (water) used for soaking the edible materials may be drained. In step 212, the edible material may be added to a mechanical grinder. In step 214, an appropriate volume of water based on weight and type of soaked edible material may be added into the mechanical grinder. In one embodiment, each of the edible material may be added into a separate grinder for grinding operation. In addition, each of the grinders may be set for grinding process. In step 216, the edible materials may be grinded for a predetermined time in the mechanical grinder to generate a batter of an optimal viscosity and granularity. In step 220 the grinder may be stopped and viscosity and granularity of the batter may be checked. The batter for first edible ingredient such as a cereal and the second edible ingredient such a pulse or a lentil or combination of both may be in the ratio of 2:1, 3:1 or 4:2:2. Many permutation and combination of cereal and pulses and/or lentils may done. Spices may be at least one of salt, chili, coriander, black pepper, fenugreek seed or powder and not limited to only these examples. One such example may be 100 grams (gms) of Rice, 50 grams of Black gram dhal, 5 mgms of salt, and 5 mgms of black pepper, 1 mgm of fenugreek seed or powder. In one embodiment, if the viscosity of the batter and the granularity is below a threshold then the step 216 is performed for additional amount of time. Insufficient granularity and viscosity 218 leads back to step 216 of grinding for a predetermined time. Once grinding process 112 is completed, the mixing process 113 is initiated. In one embodiment, the mixing process 113 is described in step 222. The generated batter may be mixed into one container to generate an admixture 226. Further, in step 224 the grounded spices may be added into the admixture. In the admixture 226, the batters are thoroughly mixed to ensure an optimal mixture for consumption. Once the admixture is generated, the admixture may be further processed in prepped material processing 103 step such as fermenting 114 as described in FIG. 3.

FIG. 3 is a flow chart illustrating a prepped material processing 103 according to one embodiment. The admixture 226 may be placed in a container. The container may be placed in a closed environment for fermentation. The fermentation process 114 includes providing an optimal temperature 310 usually between 45°-70° C. more specifically 55°-60° C., aeration 320 for a specific duration of time 350, to increase the volume of admixture 330 with an optimal consistency 340. The admixture may be stirred 360 at periodic intervals or at an appropriate time during fermentation. Keeping the admixture 226 for a specific duration of time in a closed environment may generate an admixture that is fermented. Once the fermented admixture is generated the fermented admixture is sent for packing. In a specific embodiment, if the admixture is not sufficiently fermented, the admixture is stored for an additional duration at an optimal temperature for generating the fermented admixture. The fermented admixture is sent for packaging and shipping 104.

FIG. 4 is a flow chart illustrating a packaging and shipping processes 104 according to one embodiment. The fermented admixture is sent to storage 115 in the packaging unit. In step 402, containers for storing the fermented admixture may be labeled. In step 404, measure quantity of admixture may be put into each of the labeled containers. In step 406, each of the containers may be sealed with a cover. The sealing can be done using cellophane sheets. A tiny hole can be made to vent extra build up of fermentation related gas formation. In step 408, lid may be placed and the container may be fully sealed. In step 410, a quality check may be performed and an expiration date may be stamped on each of the sealed containers. This may ensure proper storage of food products with predictable shelf life. In step 412, all the stamped containers may be stored for delivery through distribution process. The distribution process is explained in the steps below.

The stamped containers may be stored in a cold storage till the stamped containers are shipped 414. In step 416, the shipped containers may be locally stored or delivered in cold vans. Alternatively, in step 418, the shipped containers may be delivered to customers by storing in a dry ice.

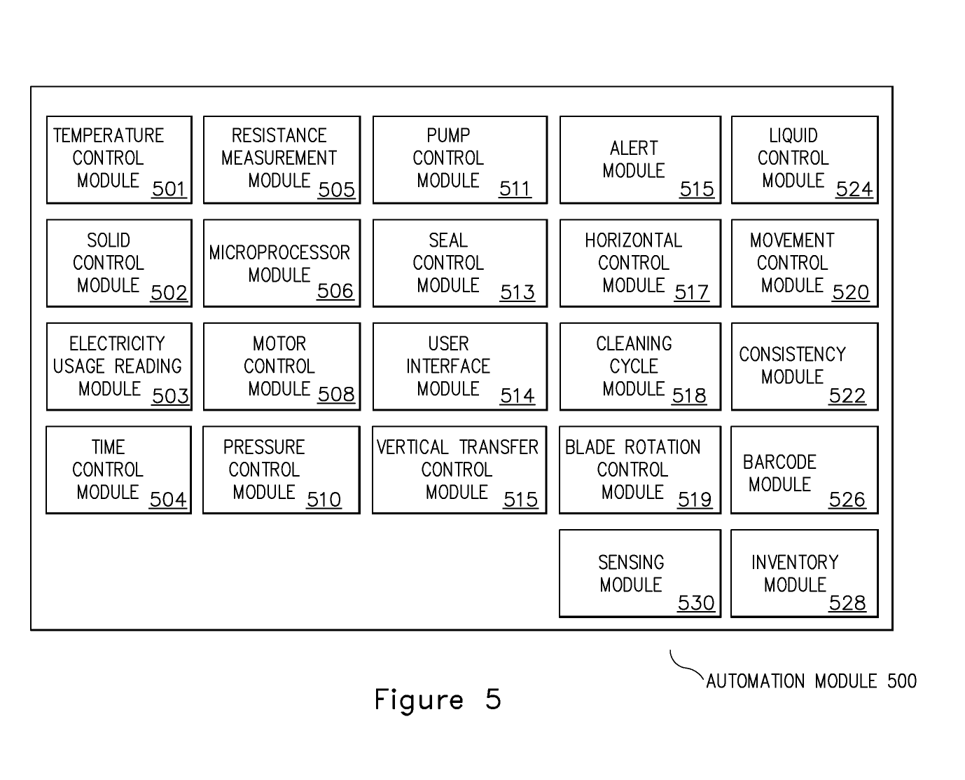

FIG. 5 illustrates an automation module 500 implemented the manufacturing unit as illustrated in FIG. 1. The Automation module 500 may include a temperature control module 501, a solid control module 502, an electricity usage reading module 503, a time control module 504, a resistance measurement module 505, a microprocessor module 506, a motor control module 508, a pressure control module 510, a pump control module 511, a seal control module 513, a user interface module 514, a horizontal control module 515, an alert module 516, a horizontal control module 517, a cleaning cycle module 518, a blade control module 519, a liquid control module 524, a movement control module 520, a consistency module 522, a robotic arm control module 515, and a barcode module 526, according to one embodiment. An alert module 515 to communicate an alert to the data processing device when there is a mismatch in requirement information and stock information. A sensing module 530

The temperature control module 501 may be configured to control temperature at various manufacturing units. The temperature control module 501 may include one or more temperature sensors being placed in different units of manufacturing unit. In one embodiment, the temperature control module 501 may control temperatures at fermentation unit for ensuring fermentation of the admixture. In addition, the temperature control module 501 will control the temperature at the storage of the sealed containers storage. The feedback to the temperature control module 501 may be provided through the temperature sensors. Based on the feedback and the preprogram programmed into the computing devices controlling the manufacturing unit, the temperature control module 501 switches on heater or cooler to maintain the temperature programmed into the computing devices controlling the manufacturing units. The solid control module 502 may be configured to dispense a measured quantity of solid edible material into the container.

The electricity usage reading module 503 may obtained electricity usage in grinding operations to determine requirement of water while performing grinding. Based on the feed back from the resistance due to increased electrical consumption and resistance measurement module 505 reading speed of the motor may be increased or decreased to achieve optimal consistency for the batter. The time control module 504 may maintain timing information in at least one of the soaking process 110, the grinding process 112 and the fermentation process 114. The time control module 504 may include timers to manage the aforementioned processes. The resistance measurement module 505 may measure the resistance measured at the grinding process 112. The resistance measurement is performed to determine the additional requirement of water and/or the motor speed needs to be adjusted. The microprocessor module 506 may control each micro controller of the manufacturing units.

The microprocessor module 506 may communicate commands to each of the micro controllers of the manufacturing unit to perform specific tasks. For example, the microprocessor module 506 may communicate a command to a mixer in the mixing unit to enable the mixer in the mixing unit to perform thorough mixing of admixture. The microprocessor module 506 also monitors coordinates and controls the functions of other modules in the automation module 500. The motor control module 508 may be configured to control the speed of motor in the grinder in the grinding process. The motor control module 508 many receive inputs from electricity usage reading module 503, the resistance measurement module 505 and the micro processor module 506 to increase or decrease the speed of motor in the grinder of the grinding module. The pressure control module 510 may be used for proving a specific pressure in the closed environment for fermentation process.

The pump control module 511 may be configured to pump sufficient amount of water into the soaking and the grinding processes. The seal control module 513 may be configured to seal the packed container such that there is no leakage of admixture from the container. The user interface module 514 may be configured for providing at least one of status update, stock update, production update, requirement update, shipping update, quality update, order, target etc. The user interface module 514 also enables an administrator of the manufacturing unit to administer various problems, such as capacity of the manufacturing unit. In addition, the user interface module 514 also enables the administrator to update the software, add software patches, change configuration, speed etc of the manufacturing unit through the automation module 500. The dispensing of the batters of the different edible materials into the mixing container may be controlled through the vertical control module 515. Vertical control module 515 may also assist in storing the containers.

The robotic arm control module 525 may be configured to control the robotic arm to perform specific tasks such as lifting and placing the containers, transferring the contents of the containers etc. The alert module 516 may be configured to generate alerts to at least one of the following situation; at least one of a machinery malfunctions, process failures, edible material stock depletion, leakage and temperature variation during fermentation, storage etc. The horizontal control module 517 is configured to control the movement of the conveyor belt to enable proper placement of containers for at least one of addition of water, mixing, packing, stacking etc. The cleaning cycle module 518 is configured to clean he edible material by washing of edible material using water before the soaking process 110. The blade rotation control module 519 is configured to control grinding blades in the grinder while performing grinding operation 112. The liquid control module 524 is configured to control the flow of water in grinding process. The movement control module 520 is configured to control the packing process by monitoring the movement of packed sealed containers in the premises of the manufacturing unit.

The consistency module 522 is configured to control the viscosity and granularity of the batter being prepared in the grinding process. The consistency module 522 may include sensors that measure viscosity and granularity of the batter in the grinding process. Based on the feedback obtained from the viscosity and granularity sensors, the consistency module 522 may enable the grinder to perform grinding for additional duration of time for generating an optimal quality of batter. The bar code module 526 is configured to generate a unique bar code for each one of the sealed containers. A sensing module 530 to weigh the storage bins to communicate a weight of the storage bins to the data processing system to determine quantity of the sealed packing container in the storage bins. An inventory module 528 to convey requirement information to the data processing device for material inventory, product inventory etc.

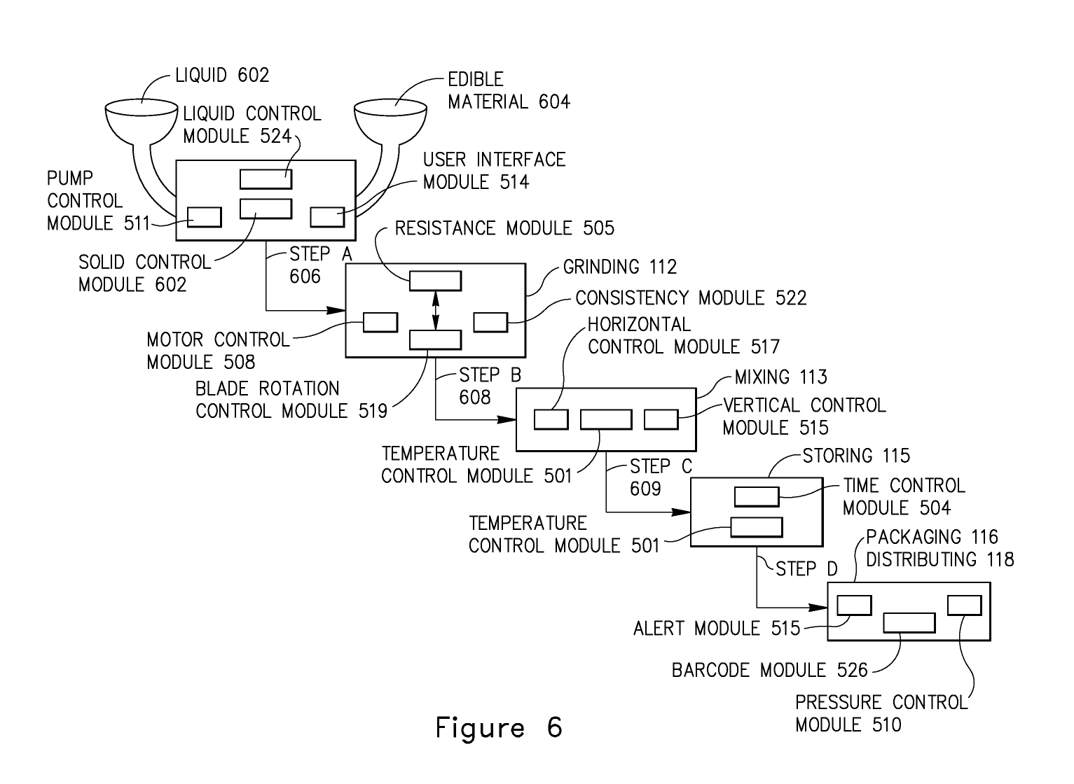

FIG. 6 is a flow diagram illustrating a generation of admixture. In one embodiment, an edible material 604 and a liquid 602, for example, water may be added into a container for cleaning and soaking. The amount of liquid being added into the container may be controlled through the pump control module 511. The solid control module 502 may be used to control the flow of solid edible materials. The user interface module 514 may be used to monitor the quantity of liquid and edible material in the container. In step A 606, the aforementioned solid edible material and the liquid after mixing, may be stored for predetermined duration of time to soak the solid edible material. Each of the measured soaked solid edible material may be put into separate grinders for grinding process 112. The grinding process may be initiated by motor control module 508. The speed of the rotation of the blades in the grinder may be observed through a blade rotation control module 619. Based on the input obtained from the blade rotation control module 519, motor control module 508 may increase or decrease the speed of motor to generate a batter at an optimal consistency. The consistency of the batter may be measured through the consistence module 522. The consistency module 522 may provide input to the motor control module 508 to perform grinding. The generated batter, in step B may be sent for mixing process 113. The batter of various edible materials may be mixed through a mixer.

The mixing container may be placed at a location to enable dispensing of batters of different edible materials into a mixing container. The movement of the conveyor carrying the mixing container may be controlled through horizontal control module 517. The dispensing of the batters of the different edible materials and grounded spices may be controlled through the vertical control module 515. The admixture generated therein the mixing process 113 may be sent for fermentation process in step C 609. The fermented batter may be stored in a closed environment, wherein the temperature may be controlled through the temperature control module 501 and the pressure may be controlled through the pressure controlled module 510. The admixture may be stored for a predetermined duration of time, wherein the time being monitored through the time control module 504. The stored admixture may be sent to packaging 116 processes and distributing process 118. The premeasured quantity of admixture may be put in standard containers for packing. The bar code module 526 may be used for placing a unique bar code on each of the packing container. The alert module 515 may be used to detect a sealed container with weight beyond the standard weight. The pressure control module 510 may be used for monitoring the pressure for lid closure. The packed and sealed containers may be placed in a cold storage for distribution.

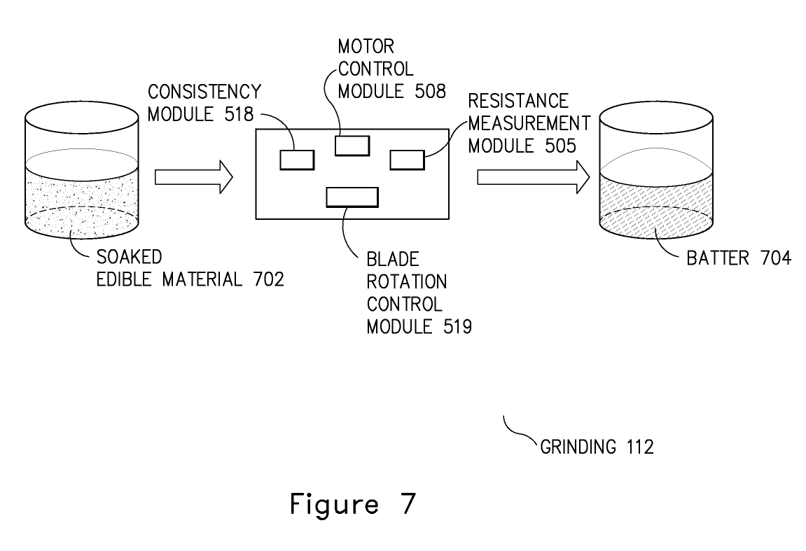

FIG. 7 is a perspective view illustrating a grinding process 112 according to one embodiment. The premeasured quantity of the soaked edible material 702 may be put in a grinder to generate a batter of optimal consistency. An appropriate amount of water may be added into the grinder to enable the grinder to generate the batter. As the grinding process 112 is initiated, the rotation of the blade may be monitored by blade rotation control module 519 to determine the speed of the rotation of blades for grinding. The resistance measurement module 505 may determine the resistance in grinding operation. Based in inputs from the resistance measurement module 505 and the blade rotation control module 519, the microprocessor module 506 may communicate a control signal to the controller of the grinder to increase or decrease the speed of the rotation of blades and motor control module 508 in the grinder to generate the batter of optimal consistency. The consistency of the batter may be checked through the consistency module 518. Based on the feedback obtained from the consistency module 518 and the microprocessor module 506 may communicate a signal to microcontroller of the grinder and motor control module 508 to perform grinding operation for additional period time or to stop the grinding operation based on the consistency of the batter.

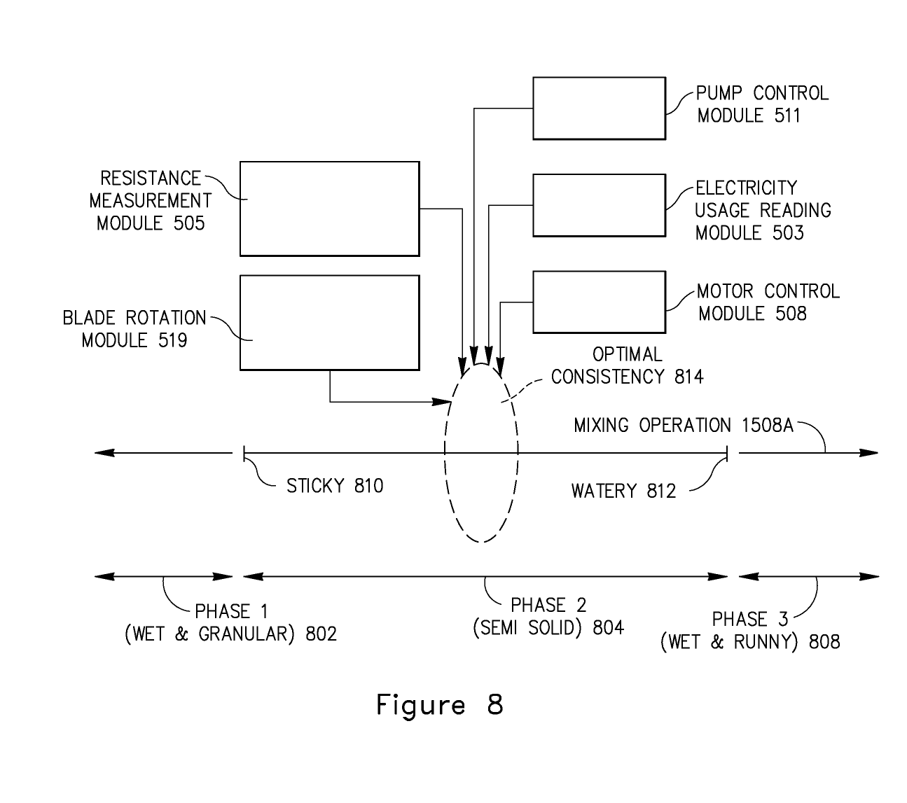

FIG. 8 illustrates various phases of preparing the admixture. A phase 1 802 is corresponds to a phase of soaking and initial grinding. A resultant of the phase 1 is a wet and granular edible materials. In the phase 1, the solid edible materials are soaked for a predetermined amount of time and placed in grinding operation. In phase 2, the grinding process 112 is continued to obtain a semi solid state of the solid edible materials. The electricity usage reading module 503, the resistance measurement module 505, the motor control module 508, the pump control module 511 and the blade rotation module 519 are used in the grinding process 112 to obtain the batter of optimal consistency with required viscosity and granularity. A phase 3 is a state where batter is in a state of semi liquid. The phase 3 is a state that is not the right consistency and viscosity. In the current invention the goal is achieve phase 2 semi solid 804 phase.

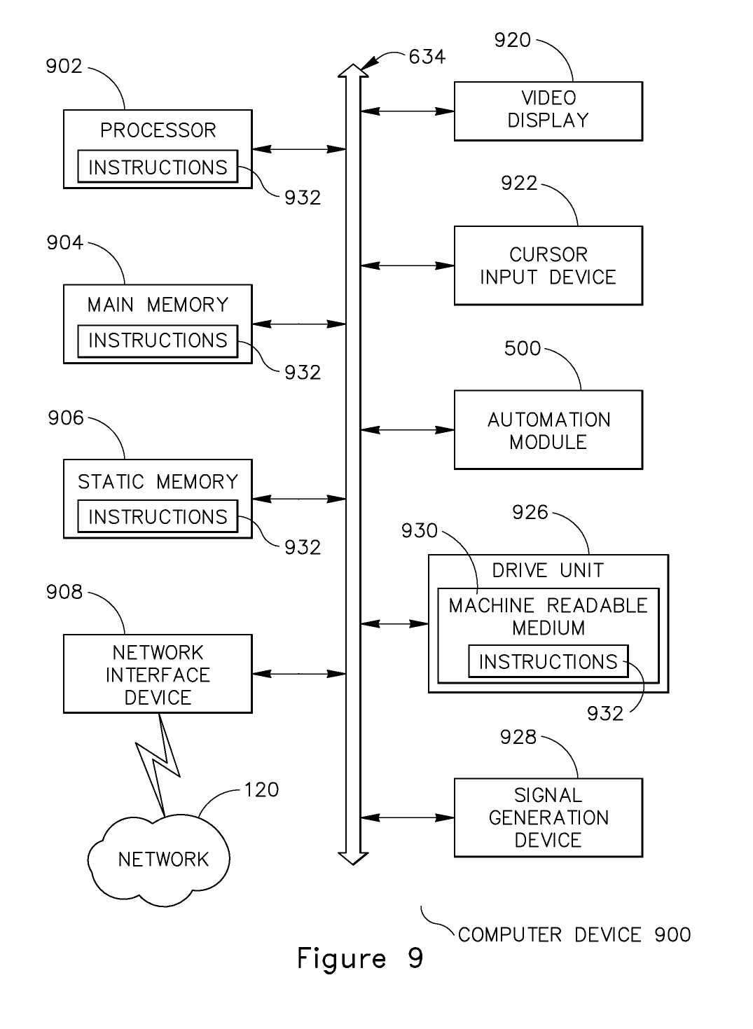

FIG. 9 is a diagrammatic system view 900 of a data processing system in which any of the embodiments disclosed herein may be performed, according to one embodiment. Particularly, the diagrammatic system view 900 of FIG. 9 illustrates a processor 902, a main memory 904, a static memory 906, a bus 908, a video display 910, an alpha-numeric input device 912, a cursor control device 914, a drive unit 916, a signal generation device 918, a network interface device 920, a machine readable medium 922, instructions 924, and a network 926, according to one embodiment.

The diagrammatic system view 900 may illustrate a physical machine, the mass migration desktop virtualization server 402, in which one or more operations disclosed herein are performed. The processor 902 may be a microprocessor, a state machine, an application specific integrated circuit, a field programmable gate array, etc. The main memory 904 may be a dynamic random access memory and/or a primary memory of a computer system.

The static memory 906 may be a hard drive, a flash drive, and/or other memory information associated with the data processing system. The bus 908 may be an interconnection between various circuits and/or structures of the data processing system. The video display 910 may provide graphical representation of information on the data processing system. The alpha-numeric input device 912 may be a keypad, a keyboard and/or any other input device of text (e.g., a special device to aid the physically handicapped).

The cursor control device 914 may be a pointing device such as a mouse. The drive unit 916 may be the hard drive, a storage system, and/or other longer term storage subsystem. The signal generation device 918 may be a bios and/or a functional operating system of the data processing system. The network interface device 920 may be a device that performs interface functions such as code conversion, protocol conversion and/or buffering required for communication to and from the network 926. The machine readable medium 922 may provide instructions 924 on which any of the methods disclosed herein may be performed. The instructions 924 may provide source code and/or data code to the processor 902 to enable any one or more operations disclosed herein.

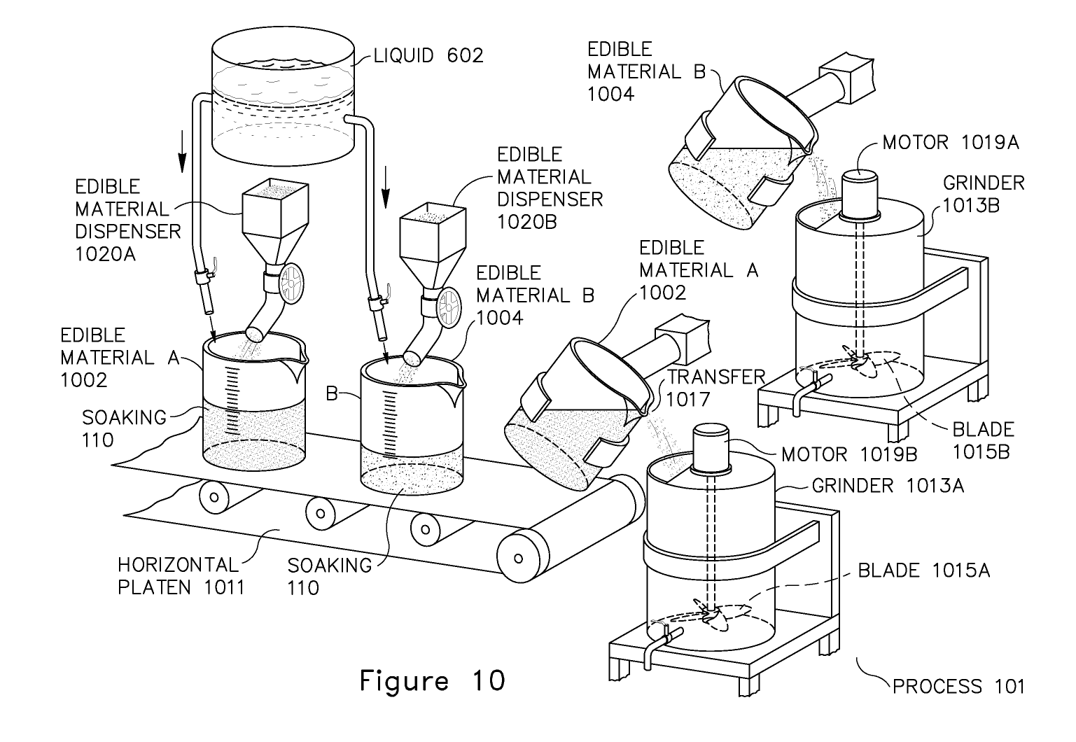

FIG. 10 is a process 101 illustrating soaking and grinding operation 112 according to one embodiment. FIG. 10 illustrates edible material dispensers 1002-1004, the liquid dispenser 602. The edible material dispensers 1002-1004 include a container and an opening. The opening area to dispense edible material may be controlled through a mechanism and the mechanism may be controlled by a motor. Each of the motor of the edible material dispenser may be configured to dispense the edible material based on the edible material in the edible material container. The quantity of the edible material to be dispensed into a specific container may be predetermined. Also, the liquid dispenser 602 may include one or more dispensing valves to dispense a liquid into the specific containers.

In one embodiment, the dispensing valve may also be controlled through the microprocessor module 506. The edible material dispensers 1002-1004 and liquid dispenser may be fixed in a location. The containers may be brought to the location of the edible materials dispensers and liquid dispensers by placing the containers on a horizontal platen. The sensors on the horizontal platen may sense the location of the containers to be displaced. The location information of the containers may be communicated to the microprocessor module 506. The microprocessor module 506 may communicate a command to the horizontal platen to move the container to the location of the dispensers, such that the edible material and the liquid may be dispensed into the containers. Further, the container comprising the edible materials and the liquid may be picked by the robotic arm and placed it in a location for soaking.

The edible material may be soaked for a predetermined duration of time. Further, the container containing the soaked edible material may be picked by the robotic arms to transfer 1017 the soaked edible materials into grinders' 1013A-1013B. In one embodiment, the soaked edible material 1002 may be dispensed into a grinder 1013Ausing a edible material dispenser 1020A and B. Similarly, the soaked edible material 1004 may be dispensed into the grinder 1013B. The grinders 1013A-1013B are machines that are designed for edible materials. Each of the grinders may include a blade designed for the purpose of grinding the edible materials. The blades 1015A-1015B of the grinder 1013A and 1013B may be controlled through a motor 1019A and 1019B respectively. The motors 1019A AND 1019B may be controlled through the motor control module 508.

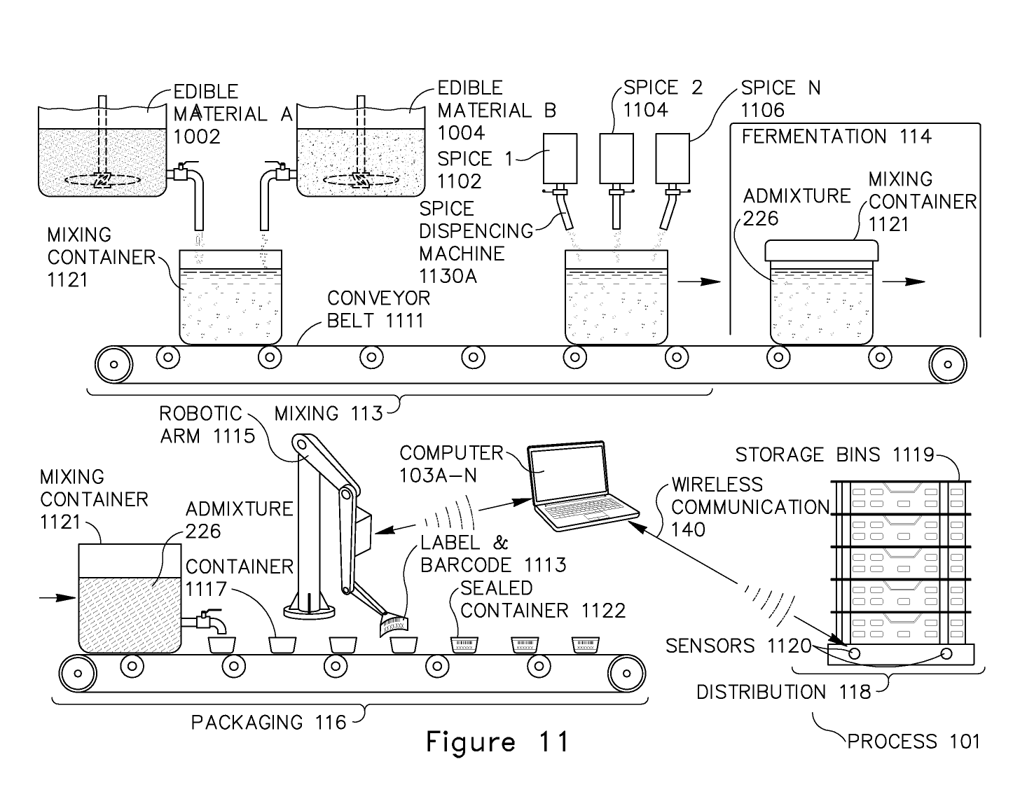

FIG. 11 is a continuation of the process 101 illustrating additional operations according to one embodiment. The batter obtained by grinding the edible material 1002 in the grinder 1013A and 1013B may be collected from the grinders in a mixing container. The mixing container is placed on a conveyor belt. The mixing container may receive the measured volume of batter from each of the grinder. The mixing container may be transported further on the conveyor belt 1111 to a location where the grounded spices can be dispensed into the mixing container 1121. In one embodiment, there may be one or more grounded spice dispensers 1102-1106 (e.g., as illustrated in the figure). The spices may be dispensed using the spice dispenser machine 1130 A-C.

Furthermore, the grounded spices many be dispensed into the mixing container. The spices may be salt, sugar, chili powder, asafetida, fenugreek seed powder, coriander leaves, curry leaves etc. Further, the mixing container may be transported to fermentation chamber. Furthermore, the mixing container may be placed in an environment at a controlled temperature and pressure for a predetermined duration of time. A measured volume of admixture may be transferred to each of the packing containers 1117. Further, a robotic arm 1115 may be used for packing, sealing and applying barcode to each of the sealed containers. The sealed containers 1122 may be placed in storage bins 1119. Weight sensors 1120 may be used to determine the total weight of the sealed containers in the storage bins 1119. In addition, radio frequency (RF) id tags may be coupled to a set of storage bins 1119 to determine the location of the storage bins being stored in the manufacturing unit.

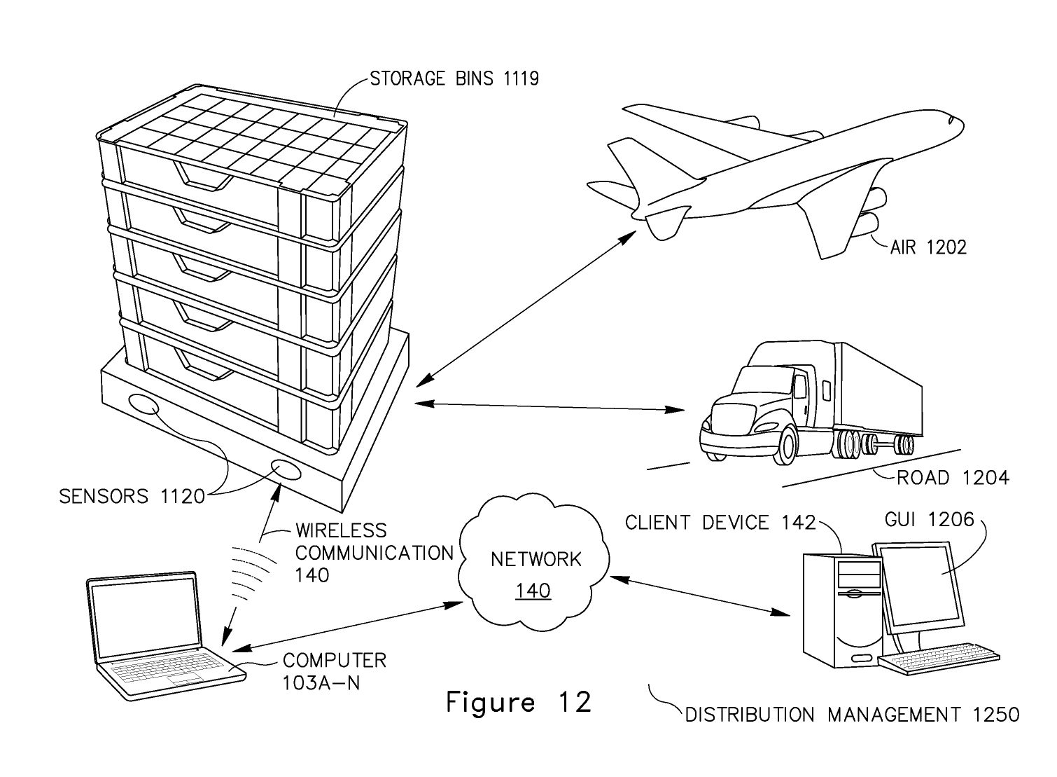

FIG. 12 illustrates a distribution management 1250 according to one embodiment. The RF id tag may be sensed in the manufacturing unit through RF id readers periodically. Further, based on the client orders, the storage bins 1119 may be shipped to client locations. The storage bins 1119 may be transported to the client locations via road 1204 or via air 1202. The RF id tags may be removed and information may be updated regarding dispatch of the storage bins 1119 from the manufacturing unit.

Although the present embodiments have been described with reference to specific example embodiments, it will be evident that various modifications and changes may be made to these embodiments without departing from the broader spirit and scope of the various embodiments. Accordingly, the specification and drawings are to be regarded in an illustrative rather than a restrictive sense.

What is claimed is:

1. A process, comprising:
soaking a pre-measured quantity of a cereal and a pulse for a pre-determined time to form a soaked cereal and a soaked pulse;
cleaning the soaked the cereal and the pulse by at least one of an automated machine and a manual process;
draining an excess water from soaked cereal and pulse;
transferring the soaked cereal and the soaked pulse to a first grinder and a second grinder;
adding pre-determined amount of water in the grinder; and
grinding the soaked cereal and soaked pulse individually in the first grinder and the second grinder to create a first batter and a second batter.

2. The process of claim 1, further comprising:
mixing the first batter and second batter to make a final batter;
adding spices to the final batter;
stirring the final batter with the spices;
fermenting the final batter at room temperature for a fixed time; and
storing the final batter at ambient temperature before packing.

3. The process of claim 2, wherein the fixed temperature is between 45-70° C., and wherein ambient temperature is between 55-60° C.

4. The process of claim 3, further comprising packing the final batter in a container designed thereof.

5. A method, comprising:
dispensing a soaked cereal for a predetermined period of time into a first grinder;
dispensing a liquid material into the first grinder comprising the soaked cereal to cover the top; and
controlling the consistency using a consistency module to generate a batter of optimal consistency through at least one of an automatic adjustment and a manual adjustment of a batter of the soaked cereal and the liquid material surrounding a blade of the apparatus based on an electrical property associated with a rotation of the blade.

6. The method of claim 5, further comprising:
dispensing a soaked pulse for a predetermined period of time into a second grinder;
dispensing the liquid material into the second grinder comprising the soaked pulse to cover the top; and
controlling a consistency of the batter using the consistency module to generate an admixture of optimal consistency through at least one of an automatic adjustment and a manual adjustment of a batter of the soaked pulse and the liquid material surrounding a blade of the apparatus based on an electrical property associated with a rotation of the blade.

7. The method of claim 6, further comprising:
grinding the soaked cereal to generate the batter of the soaked cereal;
grinding the soaked pulse to generate the batter of the soaked pulse; and
mixing the batter of the soaked cereal and the batter of the soaked pulse to generate an admixture.

8. The method of claim 7, further comprising:
monitoring the granularity and viscosity of the batter while performing grinding.

9. The method of claim 8, wherein grinding is stopped once the batter with granularity and viscosity is obtained.

10. The method of claim 7, further comprising:
mixing the batter of the soaked cereal and the batter of the soaked pulse using a ratio to make a admixture;
adding at least one spice to the admixture; and
fermenting the admixture for a predetermined amount of time.

11. A method of claim 10, wherein the cereal is at least one of be rice, wheat, semolina, barley, maize, oats, rye, sorghum, varieties of rice such as parboiled rice, wild rice and brown rice, wherein the pulse is at least one of be black gram, red gram, mung bean, chickpea and bengal gram.

12. A method of claim 10, wherein the spice is at least one of salt, sugar, chili powder, asafetida, fenugreek seed powder, coriander leaves and curry leaves.

13. The method of claim 10, further comprising:
removing the excess air from a fermented admixture;
pouring the admixture in a container;
labeling the container;
stamping the container with an expiration date; and
storing the container till it is ready for a shipment.

14. A system comprising:
at least one dispenser controlled through a data processing device to dispense a predetermined quantity of an edible material into at least one of a container;
a liquid dispenser controlled through the data processing device to dispense a predetermined quantity of liquid into the at least one of the container to soak the at least one of the edible material for a predetermined duration of time;
a first conveyor machine controlled through the data processing device to place the at least one container into a position to enable the at least one dispensers and the liquid dispenser to dispense into the container and to place the at least one container in a position to enable a first robotic arm to transfer the soaked edible materials in at least one of the containers into at least a grinder;
the first robotic arm to hold the container with appropriate pressure to transfer the soaked edible material into the at least one of the grinder; and
the at least one of the grinder controlled through the data processing device to grind at least one of the soaked edible material into a batter of optimal consistency.

15. The system of claim 9, further comprising:
at least one spice dispensing machine to dispense grounded spices into the batter of optimal consistency;
at least one mixing apparatus to mix the batter of the edible materials and the grounded spices in a mixing container; wherein the batter is at least one of a cereal batter, a pulse batter and a pulse batter; and
a second robotic arm to place the mixing container into a fermentation chamber to perform a fermentation process.

16. The system of claim 10, further comprising:
the second robotic arm to place the mixing container from the fermentation chamber at a predetermined location to enable dispensing of the mixed batter into a packing container on the second conveyor machine;
a third robotic arm controlled through the data processing device to seal the packing container and to apply a label and barcode on the sealed packing container;
a fourth robotic arm controlled through the data processing device to place the sealed packing container into storage bin to be distributed through a distribution system; and
a sensing module to weigh the storage bins to communicate a weight of the storage bins to the data processing system to determine quantity of the sealed packing container in the storage bins.

17. The system of claim 11, wherein the data processing device enabled through a set of codes executed through a processor of the data processing device to manage the system.

18. The system of claim 10, wherein the fermentation process is performed in room temperature.

19. The system of claim 9, wherein the data processing device to communicate with the at least one edible material dispenser, a liquid dispenser, a horizontal platen, the first robotic arm the at least one grinder, at least one spice dispensing machine, at least one mixing apparatus, a second robotic arm, the mixing machine, a third robotic arm, a fourth robotic arm, and a sensing module through wired or wireless communication.

20. The system of claim 12, further comprising:
an inventory module to convey requirement information to the data processing device;
a database to maintain information associated with at least one of stock of the edible material, the liquid, the grounded spice, and packing container; and
an alert module to communicate an alert to the data processing device when there is a mismatch in requirement information and stock information.
